(19) United States
(12) Patent Application Publication
(10) Pub. No.: US 2005/0215933 A1
(43) Pub. Date: Sep. 29, 2005

(54) Ulnar styloid brace

(76) Inventors: Jeffrey Stearns, Hopatcong, NJ (US); James D. Israel, Somerset, NJ (US)

Correspondence Address: HARNESS, DICKEY & PIERCE, P.L.C., P.O. BOX 828, BLOOMFIELD HILLS, MI, 48303, US

(21) Appl. No.: 10/812,381

(22) Filed: Mar. 29, 2004

(51) Int. Cl. A61F 13/00

(52) U.S. Cl. 602/57

(57) ABSTRACT

A brace to be worn over the wrist for preventing and reducing pain and discomfort associated with the ulnar styloid of the wrist. The brace includes a band circumferentially fitted around the wrist. The band has an opening positioned to circumferentially surround the ulnar styloid. A pressure ring is attached to the opening, so that when the band is fittingly secured around the wrist with the ulnar styloid protruding through the opening, the pressure ring exerts controlled pressure to tissues associated with the ulnar styloid.

The wrist is one of the most common sites for upper-extremity injuries and pain or discomfort experienced during sporting activities, which can include swinging motions in common leisure sports such as basketball, soccer, mountain biking, skiing, golf, and racket sports.

As shown in FIGS. 1 and 2, the wrist 90 includes eight carpal bones 92, the ulna 94, the ulnar styloid 96, the radius 98, and various soft tissue structures, including the extensor carpi ulnaris (ECU) tendon 80 and the triangular fibrocartilage complex (TFCC) 82. One of the manifestations of wrist-related injury or pathology is pain or discomfort that occurs in the ulnar styloid region of the wrist. Typically, such discomfort results from one or more of the following conditions: a TFCC tear, ECU tendon or sheath related problems, including ECU tenosynovitis and subluxation, ulnar styloid nonunion, and other wrist and forearm pathologies of undetermined nature. Pain can result from the sixth dorsal compartment of the ECU tendon 80 pressing on the ulnar nerve 84.

Although various wrist supports are commercially available for various conditions, there is currently no support device that specifically addresses ulnar styloid problems.

### SUMMARY

The present teachings provide a brace to be worn over the wrist for preventing and reducing pain and discomfort associated with the ulnar styloid of the wrist. The brace includes a band circumferentially fitted around the wrist. The band has an opening positioned to circumferentially surround the ulnar styloid. A pressure ring is attached to the opening, so that when the band is fittingly secured around the wrist with the ulnar styloid protruding through the opening, the pressure ring exerts controlled pressure to tissues associated with the ulnar styloid.

The present teachings also provide a method for preventing and reducing pain and discomfort associated with the ulnar styloid of the wrist. The method includes positioning an adjustable band having a pressure ring around the wrist, placing the pressure ring over and around the ulnar styloid of the wrist, and controlling the pressure of the pressure ring over the ulnar styloid.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a simplified representation of the various bones of a wrist;

FIG. 2 is a transverse section of the wrist;

FIG. 3 is a top perspective view of a brace for the ulnar styloid of a wrist according to the present teachings;

FIG. 3(a) is a cross-sectional view of a brace according to the present teachings;

FIG. 4 is an assembly view of the brace of FIG. 3; and

FIGS. 5(a), 5(b) and 5(c) illustrate how to use the brace of FIG. 3.

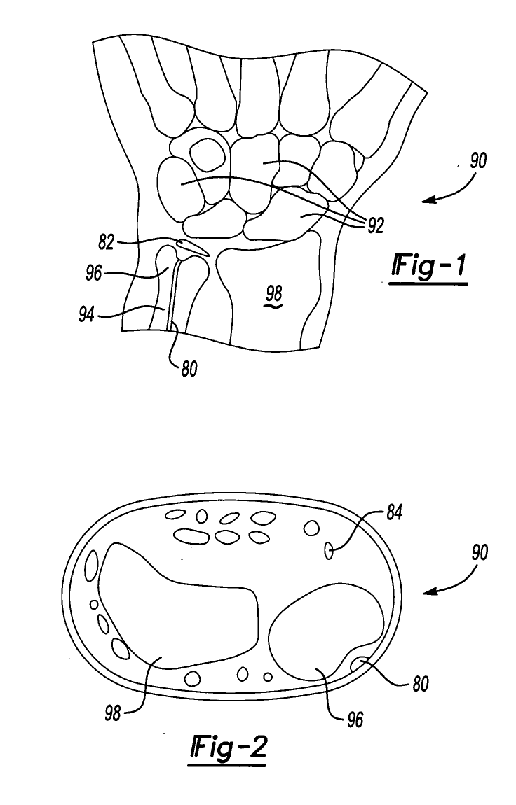

### DETAILED DESCRIPTION

The following description is merely exemplary in nature and is in no way intended to limit the invention, its application, or uses.

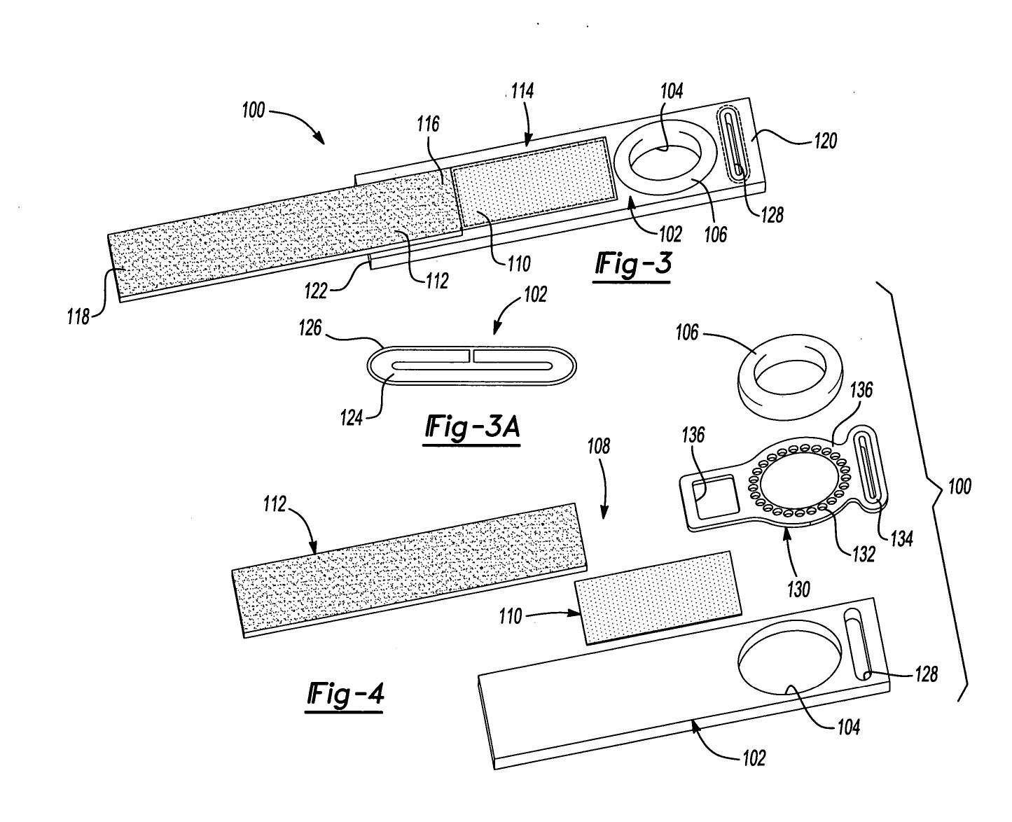

Referring to FIGS. 3 and 4, an exemplary brace 100 to be worn on the wrist 90 for preventing or relieving pain and discomfort associated with the region of the ulnar styloid 96 is shown. The brace 100 includes a band 102 with an opening 104. A pressure ring 106 is overmolded or attached by other known means onto the opening 104. The pressure ring 106 can be made of an elastomer, such as medical grade silicone with a 20-30 durometer Shore A, and is shaped to go over and surround the ulnar styloid 96. The undeformed shape of the pressure ring 106 can be, for example, elliptical, oval, ovoid or other suitable shape, and can be provided in different sizes for different size wrists. The brace 100 can be worn on the right or left wrist, and in particular during activities that cause or exacerbate ulnar styloid pain, such as the swinging motions involved in golf, baseball, tennis, etc.

The band 102 can be adjustable for providing controlled pressure on the ECU tendon 80 to hold the ECU tendon 80 in place and prevent the band 102 from pressing on the ulnar nerve 84. A hook-and-loop fastener 108 having a first strap 110 and a second strap 112 can be used to adjust the band 102. One of the straps 110, 112 is a hook strap and the other is a loop strap. The hook and loop straps 110, 112 are attached onto the top surface 114 of the band 102, which is the surface opposite to the surface that comes in contact with the wrist 90. The hook and loop straps 110, 112 can be sewn or glued or attached by known methods on the top surface 114 of the band 102. The first strap 110 is positioned adjacent the pressure ring 106. The second strap 112 is placed next to the first strap 110 and has a first end 116 attached to the band 102 and a second free end 118. The length of the second strap 112 can be shortened by cutting the second strap 112 from the free end 118. The band 102 has a first end 120 adjacent to the pressure ring 106 and a second end 122 which can also be cut to fit the circumference of the wrist 90.

The band 102 includes an elongated slot 128 between the pressure ring 106 and the first end 120. The slot 128 is shaped to accommodate the width of the second strap 112, which can be inserted through the slot 128, folded over and attached to the first strap 110.

Referring to FIG. 3a, the band 102 can include padding 124. The band 102 can be made, for example, from a padded strap of material that is folded and attached to form a flattened tube, such that the padding 124 is completely enclosed by an external material 126. The external material 126 can be selected from materials that are naturally or artificially hypoallergenic and non-abrasive against the skin of the wrist 90. The external material 126 can also be selected to provide a desired combination of smoothness such that the brace 100 can easily slide over the wrist 90 before fastening, while providing sufficient friction such that the brace 100 can be fastened around the wrist 90 to control the pressure provided by the pressure ring 106.

The band 102 can be reinforced with a frame 130, which can be sewn as an insert inside the exemplary tubular band 102 shown in FIG. 3(a), or otherwise incorporated into the band 102 by known methods. The frame 130 can be made of plastic or other material that can increase the stiffness of the band 102 without adding much weight, such that the brace 100 remains flexible while retaining its shape over extended use, after washing, etc. The frame 130 includes an annular portion 132 that reinforces the opening 104 of the band 102, and a D-ring 134 that reinforces the slot 128. Other cutouts 136 can be provided on the frame 130 to reduce the weight of the frame 130 and increase its flexibility.

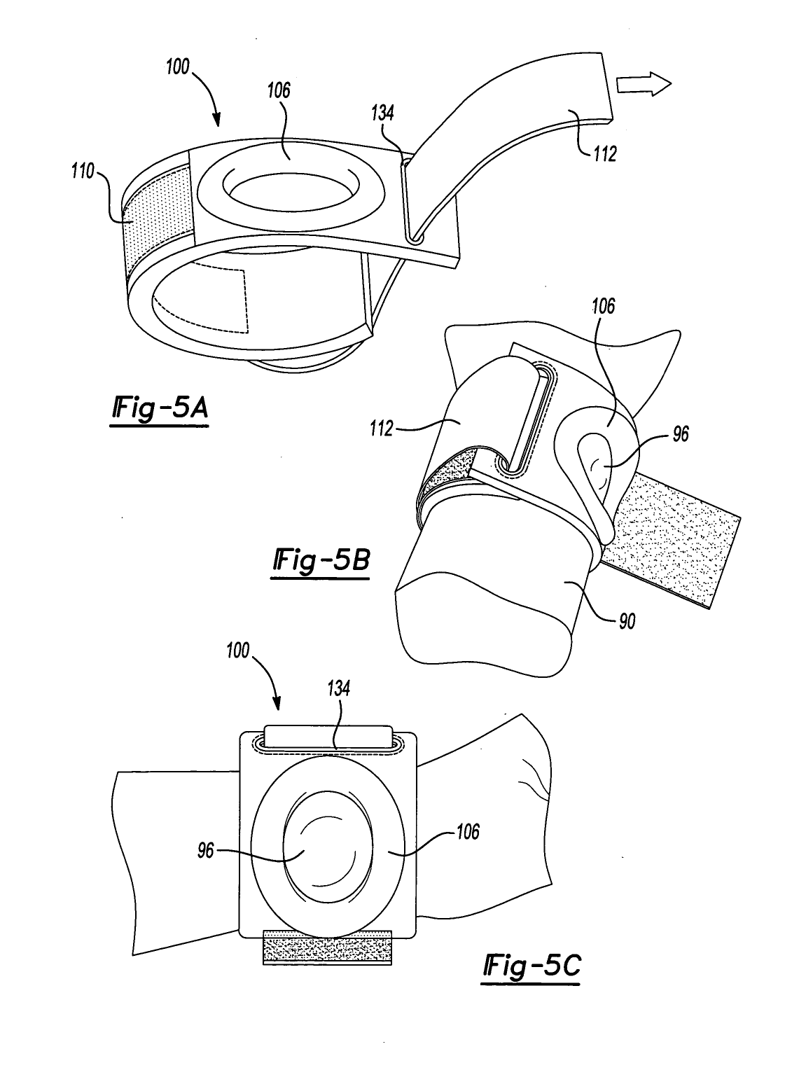

Referring to FIGS. 5(a) to 5(c), the brace 100 can be presented for use with the second strap 112 already loosely looped through the D-ring 134. The second strap 112 is be loosened as needed to slip the brace 100 over the affected wrist 90, as shown in FIG. 5(a). The pressure ring 106 is placed over the ulnar styloid 96 with the D-ring 134 on top of the wrist 90, as shown in FIG. 5(b). The second strap 112 is tightened to a desired tension and fastened to the first strap 110 to provide controlled pressure on the ulnar styloid area through the pressure ring 106. After adjustment, any excess length of the second strap 112 can be trimmed away. Excess length of the band 102 can also be cut such that the two ends 120, 122 of the band 102 meet around the wrist 90.

The brace 100 was experimentally tested on various patients suffering from pain in the ulnar styloid region of the wrist at the Curtis National Hand Center of the Union Memorial Hospital in Baltimore, Md. The brace 100 was well tolerated and found to reduce pain of the ulnar styloid region during activities that cause or aggravate such pain.

While particular embodiments have been described in the specification and illustrated in the drawings, it will be understood by those skilled in the art that various changes may be made and equivalents may be substituted for elements thereof without departing from the scope of the invention as defined in the claims. In addition, many modifications may be made to adapt a particular situation or material to the present teachings without departing from the essential scope thereof. Therefore, it is intended that the present teachings are not be limited to the particular embodiments illustrated by the drawings and described in the specification, but that the present teachings will include any embodiments falling within the foregoing description and the appended claims.

1. A brace to be worn over the wrist for preventing and reducing pain and discomfort associated with the ulnar styloid of the wrist, the brace comprising:
a band circumferentially fitted around the wrist, the band having an opening positioned to circumferentially surround the ulnar styloid; and
a pressure ring attached to the opening, so that when the band is fittingly secured around the wrist with the ulnar styloid protruding through the opening, the pressure ring exerts controlled pressure to tissues associated with the ulnar styloid.

2. The brace of claim 1, wherein the pressure ring stabilizes the ECU tendon and prevents the tendon from impinging on the ulnar nerve.

3. The brace of claim 1, wherein the band is adjustable for providing controlled pressure.

4. The brace of claim 3, wherein the band includes a strap extending from the band and securable on the band with a hook-and-loop fastener.

5. The brace of claim 4, wherein the band includes a slot for receiving the strap.

6. The brace of claim 5, wherein the slot is defined by a D-ring.

7. The brace of claim 5, wherein the band includes an insert defining the D-ring.

8. The brace of claim 7, wherein the insert is a frame reinforcing the opening.

9. The brace of claim 6, wherein the pressure ring is an elastomer.

10. The brace of claim 9, wherein the elastomer is medical grade silicone.

11. The brace of claim 9, wherein the pressure ring is deformable to a shape defined by the ulnar styloid upon adjusting the band.

12. The brace of claim 1, wherein the band includes padding.

13. The brace of claim 1, wherein the pressure ring is oval.

14. A method for preventing and reducing of pain and discomfort associated with the ulnar styloid of the wrist, the method comprising:
positioning an adjustable band having a pressure ring around the wrist;
placing the pressure ring over and around the ulnar styloid of the wrist; and
controlling the pressure of the pressure ring over the ulnar styloid.

15. The method of claim 14, wherein controlling the pressure comprises adjusting the band.

16. The method of claim 15, wherein adjusting the band comprises tightening the band over the wrist.

17. The method of claim 16, wherein tightening the band comprises:
inserting a strap having a first end attached to the band through a D-ring defined in the band;
pulling a second end of the strap through and over the D-ring; and
securing the strap to the band.

18. The method of claim 17, wherein securing the strap to the band includes attaching the strap to the band with a hook-and-loop fastener.
